(19) United States
(12) Patent Application Publication
(10) Pub. No.: US 2019/0259497 A1
(43) Pub. Date: Aug. 22, 2019

(54) METHOD AND COMPUTER FOR OPTIMIZED DISTRIBUTION OF PERSONS AMONG EXAMINATION FACILITIES

(71) Applicant: Siemens Healthcare GmbH, Erlangen (DE)

(72) Inventor: Mario Zeller, Erlangen (DE)

(73) Assignee: Siemens Healthcare GmbH, Erlangen (DE)

(21) Appl. No.: 16/276,709

(22) Filed: Feb. 15, 2019

(30) Foreign Application Priority Data
Feb. 19, 2018 (EP) 18157444

(51) Int. Cl. G16H 40/20; G06Q 10/10; G16H 10/60

(52) U.S. Cl. CPC G16H 40/20; G16H 10/60; G06Q 10/1093

(57) ABSTRACT

In a method and computer for automatic allocation of examination subjects to an examination facility among a number of medical examination facilities, first data are accessed by the computer, the first data being subject-related data and representing more detailed information about the examination subject. Second data are also accessed by the computer, the second data being operator-related data that indicate how many operators are working at or are available at the multiple medical examination facilities. The computer also accesses third data, the third data being examination facility-related data and representing information about the examination facility itself. Examination subjects are allocated by the computer to one of the examination facilities, taking into account the first data, the second data, and the third data.

### BACKGROUND OF THE INVENTION

### Field of the Invention

The present invention concerns a method for the automatic allocation of examination subjects by a computer to an examination facility among a number of medical examination facilities. The present invention furthermore concerns a medical examination apparatus and a non-transitory, computer-readable data storage medium encoded with programming instructions that implement a method of the aforementioned type.

### Description of the Prior Art

In medical examinations, in particular in examinations with an imaging apparatus, in an emergency it may be important to be able to conduct an examination for a patient as quickly as possible using the correct examination facility. For instance, the correct placement of obese patients with multiple injuries or multi-morbid patients on an examination bed is often time-consuming and challenging if the respective imaging apparatus is staffed by only a single operator, for example. In the case of emergency patients, this can lead to unacceptable delays. While it has been possible to continually further shorten the imaging part of the procedure with various techniques to accelerate the process, in the case of MRI units in particular, a not inconsiderable part of the examination time is occupied by the positioning of the examination subject and preparation for the measurement. This also includes the positioning of the necessary local coils, which have to be changed between examinations of various persons. For examinations with an examination apparatus, an optimized progression can sometimes be achieved, for example, by conducting similar or identical examinations in a chronological sequence. It is possible, with MRI apparatuses, to plan examinations of one anatomical region either exclusively, or preferably in a chronological sequence, in order to avoid coil changeover times. However, this does not solve the problem when emergencies occur.

U.S. Pat. No. 7,562,026 B2 discloses a radiology information system that allows dynamic patient planning. Yet even this does not lead to a satisfactory result.

### SUMMARY OF THE INVENTION

An object of the present invention is to improve methods for the automatic allocation of examination subjects among examination facilities.

A first aspect of the invention is a method for the automatic allocation of examination subjects to an examination facility among a number of medical examination facilities at a medical examination site, the method being implemented by a control computer. First data are acquired, the first data being subject-related data comprising more detailed information about the examination subject. Furthermore, second data are acquired, the second data being operator-related data indicating at least how many operators are working on or are available for a number of medical examination facilities. Furthermore, third data are acquired, the third data representing examination facility-related data and information relating to the examination facility itself. Subsequently, the examination subjects are respectively assigned to one of the examination facilities, taking into account the first data, the second data, and the third data.

By using the various data, that is, taking into account the subject-related data pertaining to the examination subject, taking into account the operator-related data and the examination facility-related data, it is possible to select the optimum examination facilities for an examination subject and allocate said subject to this examination facility. With these three different types of data, a comprehensive overview can be achieved, as to which examination facility among the various examination facilities is the best for a subject who is to be examined. In particular, where time is short in emergency cases, the optimum examination facility can be selected, for example the one where the necessary operating personnel are available for the examination that has to be carried out. The possible examination facilities may be of the same type, or may differ in their configuration, performance etc. The examination facilities may all be MRI units, but they may be units with different designs of magnets, with different field strengths or configurations. There may also be different CT, PET or other imaging facilities, or operating tables with different configurations.

It is possible, for example, to acquire the second data, that is, the operator-related data from duty rosters listing the possible operators, with the second data comprising current data indicating which and how many operators are currently available at the individual examination facilities, with the second data likewise comprising future data that indicates which and how many persons are available at the individual examination facilities in a time period of days, weeks, or months. The allocation of examination subjects is then achieved taking into account the current or future data.

Furthermore, it is possible for the first data, the subject-related data, to represent one of the following subject-related items of information. First information, which is contained in the first data, may be information about the urgency of the examination to be carried out. A further item of information in the first data can be a weight of the examination subject who is being examined. Furthermore, information about the height of the examination subject may be included in the first data. In addition, information about possible allergies in the examination subject or for example about contrast agent intolerance may be provided in the first data. The first data may further include information about the anatomical region to be examined, information as to whether a contrast agent will be necessary in the examination, or information as to whether the examination subject is claustrophobic, for example. Some of these items of information can be provided and used as first data during allocation to the various examination facilities.

This first data may be available with the use of a patient database that is provided in the facility such as the hospital, for example, in which the number of examination facilities are located.

A further option for acquiring the second data, the operator-related data, consists in said second date being acquired from login details, with which the operators log in at the medical examination facilities for personal identification. This information can be acquired on the individual examination facilities, also with the aid of an RFID device with which an operator identifies himself or herself on the examination facilities. In this way, current states of occupation of the facilities by operating personnel can be determined.

The examination facility can be an imaging apparatus, an MRI apparatus for example, but it is also possible for it to be a CT unit or any other imaging apparatus. The third data, which provides information relating to the examination facility, can also be acquired from the image data generated by examination subjects who were recently examined in the imaging apparatus. In particular, in MRI units, information can be acquired, for example, about a table position, the coils currently being used, the type of examination currently being used, the duration of the remainder of the examination, the bore diameter of the MRI unit, the availability of various coils etc.

For example, it is possible for the various examination subjects to be allocated to the various MRI units in such a way that in at least one of the MRI units, a number of changes of the receiving coils is minimized in the examination of various examination subjects.

For MRI units, the third data can also be used to obtain information about the size of the bore in the MRI unit, such that examination subjects with claustrophobia are for example allocated to the MRI apparatus (scanner) that has a larger bore than other MRI units. This likewise applies to obese examination subjects.

The invention is not restricted to MRI units or medical imaging facilities, however. The examination facility can also be extended to the area of surgical interventions, the examination facility then being, for instance, an operating room facility or a cardiac catheter facility.

The control computer can also have access to training data that provide information as to with which first, second and third data from previous examinations of various persons the various examination subjects were allocated to the examination facility. The control computer can then learn the allocation of examination subjects to an examination facility by means of this training data.

Furthermore, the allocation of examination subjects to one of the examination facilities can be achieved using pre-set rules that contain information regarding to which examination facilities an examination subject is allocated based on the first, second and third data. It is possible with these pre-set rules to establish how the allocation to the individual examination facilities can be achieved for the operators of the examination facilities.

Furthermore, the invention encompasses the control or scheduling computer at a medical examination site that is configured for the automatic allocation of examination subjects to an examination facility among a number of medical examination facilities at the site. The control computer has a memory and at least one processor, which can execute control commands stored in the memory. In the execution of the control commands, the control computer is configured for the processor to carry out the method described above.

The present invention also encompasses a non-transitory, computer-readable data storage medium encoded with programming instructions that, when the storage medium is loaded into a scheduling computer for multiple medical examination facilities, cause the scheduling computer to operate so as to implement any or all embodiments of the method according to the invention, as described above.

The features described above and features that are described hereinafter can not only be used in the corresponding combinations that have been described explicitly, but also in other combinations, insofar as it is not explicitly stated otherwise, without departing from the scope of the present invention.

### BRIEF DESCRIPTION OF THE DRAWINGS

FIG. 1 is a block diagram of a control computer that is configured to automatically assign examination subjects to various examination facilities, according to the invention.

FIG. 2 is a flowchart of the basic steps for assigning the examination subjects to the examination facilities, according to the invention.

### DESCRIPTION OF THE PREFERRED EMBODIMENTS

The present invention is then described in greater detail by means of preferred embodiments with reference to the drawings. In the figures the same reference characters denote the same or similar elements. Furthermore, the figures are diagrammatic representations of various embodiments. The elements shown in the figures are not necessarily shown to scale. The elements shown in the figures are rather reproduced such that their function and the purpose are comprehensible to a person skilled in the art. The connections between functional units or other elements that are shown in the figures can also be implemented as an indirect connection, a connection being wireless or wired. Functional units can be implemented as hardware, software, firmware or a combination thereof.

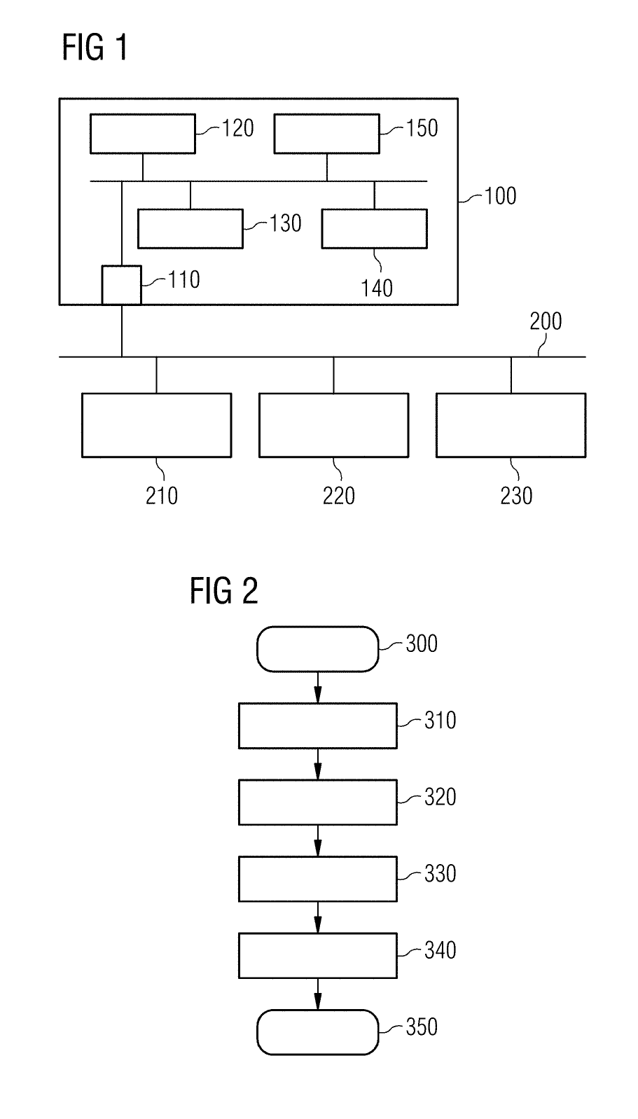

FIG. 1 is a block diagram of a control computer 100 that is configured to automatically assign examination subjects or patients to one among a plurality of medical examination facilities. The control computer 100 has an interface 110, with which a communication with other units is facilitated, for example with a first database 210, a second database 220 or a third database 230, which are further explained in detail below. Via the interface 110, data or control commands are received or transmitted. Furthermore, the control computer 100 has a processor 120, formed by one or a number of sub-processors that can execute control commands that are stored in a memory 130. Furthermore, the control computer 100 has an input unit 140 and a display unit 150. Via the input unit 140, an operator of the control computer 100 can operate the control computer 100, and via the display unit 150, information can be displayed, for example the specific allocation to the individual examination facilities. The control computer 100 is connected via a bus system 200 to the first database 210, which contains first data, which are subject-related data and represent more detailed information about the examination subject. The first database 210 can be a hospital information system HIS, for example. In addition, the control computer 100 can access a second database 220, via which information about the operators who are working on the individual examination facilities can be obtained. The examination facilities can be imaging apparatus, such as MRI units, computed tomography systems, PET systems, or the examination facilities can be interventional units such as operating room units or cardiac catheter facilities. The second database 220 can contain, for example, the duty schedules of the individual operators and physicians on duty for the individual examination facilities. Furthermore, it is possible that the second database 220 retrieves information from a login system, with which the individual operators on the examination facilities log in, for example, via RFID-Tags or suchlike. Furthermore, a third database 230 can be accessed, which contains examination facility-related data. The third data can be acquired, for example, via an interface provided for this purpose, which is present in an examination facility itself, or in the case of imaging apparatus, the information stored in the images such as, for example, the meta-information and the headers, can be examined in order to acquire information about the current status of the imaging apparatus. Since this data is available in a more or less standardized format, for example in a DICOM format, it is easy to acquire the necessary information from this data.

In the example shown in FIG. 1, the three databases 210, 220 and 230 are separate databases. Of course, this first, second and third data can also be stored in a single database, and it is possible for the data contained therein also to be stored in the control computer 100 itself, for example in the memory 130.

The apparatus shown in FIG. 1 now has access to multiple types of data, such as the current occupancy of the individual examination facilities by the operating personnel. Information is available about the current status and the basic data relating to the examination facility, in the case of an MRI unit, for example, the table position, the coils currently being used, the type of examination currently in progress, the duration of the remainder of the examination, the diameter of the bore, the possible movability of the table, the availability of coils, etc. Furthermore, data are available about the examination subject who is to be examined. This data can include, for example, weight, size of the examination region, contrast agent intolerance, susceptibility to claustrophobia or information about whether contrast agent is necessary, for example, and how urgent the necessary examination is.

Based on this data, allocation of the examination subjects to the various examination facilities then ensues using rules that are stored in the memory 130 or through a machine learning method, which has learned an optimum patient allocation based on, for example, training data sets relating to past examinations. If, for example, two, three, or four different MRI units are available, then to avoid frequent coil-changing, examinations of similar body regions can be carried out together on an MRI unit with few coil changes. Furthermore, this makes it possible to allocate obese or claustrophobic examination subjects to the MRI units that have a larger bore than other MRI units. This may avoid time-consuming rescheduling where an examination is refused or is not possible for the examination subject. Furthermore, for persons who require more time because they are either obese or have a very poor general state of health, it is possible to plan examinations at the examination facilities where more operating personnel are available. Urgent examinations can be carried out on a device that is currently available or vacant and potential subsequent examinations can likewise be redistributed. In addition, manual rescheduling of examination subjects is also possible, for example, via the input 140. Since user-defined rules can also be stored in the memory 130, these can be taken into account in the automatic allocation by the control computer 100. The system shown in FIG. 1 can also be directly connected to the individual examination facilities via the bus 200, either to obtain information from there about the examination facilities or to report the allocation of the examination subjects to the examination facilities that has been calculated.

FIG. 2 summarizes the method in diagrammatic form. The method starts in a step 300 and in a step 310 there ensues the acquisition of the first data, which is subject-related data and comprises more detailed information about the examination subject, said data being obtained from the first database 210. In a further step 320, the operator-related data is acquired, through which it is specified how many operators or which operators are available at the individual examination facilities. In a third step 330, the examination facility-related data is acquired from the database 320 or directly via the examination facilities themselves. With these three sets of data it is then possible in step 340 to optimally assign an examination subject to one of the examination facilities. The method ends in step 350.

The method described above allows an intelligent distribution of examination subjects to the respective examination facilities with all the necessary data being included. It has the advantage that, in an emergency, the examination subject can be allocated to the correct examination facility. Furthermore, an increased throughput is possible due to less rescheduling and an optimum use of the operators. This also leads to increased satisfaction among the operators and in the subjects examined due to the shorter waiting time and the correct examination facility. In addition, the planning personnel have a lighter load since fewer manual planning operations have to be carried out.

Although modifications and changes may be suggested by those skilled in the art, it is the intention of the Applicant to embody within the patent warranted hereon all changes and modifications as reasonably and properly come within the scope of the Applicant's contribution to the art.

1. A method for automatic allocation of examination subjects by a computer of a medical examination site, which has a plurality of medical examination facilities, said method comprising:
from a computer situated at said medical examination site, accessing first data from a database, said first data being subject-related data that represent detailed information about an examination subject to be examined using at least one of said plurality of medical examination facilities at said site;
from said computer, accessing said database to acquire second data, said second data being operator-related data representing a number of operators working at or available at said plurality of medical examination facilities;
from said computer, accessing said database to acquire third data, said third data being medical examination facility-related data representing information about respective medical examination facilities in said plurality of medical examination facilities; and
in said computer, executing an allocation algorithm to respectively allocate individual examination subjects to respective medical examination facilities in said plurality of medical examination facilities, dependent on said first data, said second data and said third data, and making a result of execution of said allocation algorithm available from said computer as an output in electronic form.

2. A method as claimed in claim 1 comprising acquiring said second data from duty rosters for said operators, said second data comprising current data representing which operators and how many operators are currently available at respective medical examination facilities in said plurality of medical examination facilities, and comprising future data representing which operators and how many operators will be available at respective medical examination facilities at a future point in time, and executing said allocation algorithm dependent on both said current data and said future data comprised in said second data.

3. A method as claimed in claim 1 wherein said first data comprise information selected from the group consisting of information representing a weight of the examination subject, information representing a height of the examination subject, information representing contrast agent intolerance of the examination subject, information representing an anatomical region of the examination subject to be examined, information representing a priority of the examination to be implemented for a respective examination subject, information representing whether a contrast agent is necessary for examination of the examination subject;
information representing whether the examination subject is claustrophobic, and information representing allergies of the examination subject.

4. A method as claimed in claim 1 comprising acquiring said first data from a patient database at said site.

5. A method as claimed in claim 1 comprising acquiring said second data from login entries made by respective operators logging-in to respective medical examination facilities in said plurality of medical examination facilities.

6. A method as claimed in claim 1 wherein said first data comprise information representing a weight of the examination subject, and comprising executing said allocation algorithm to allocate the examination subject to a respective medical examination facility, in said plurality of medical examination facilities, that can accommodate an examination subject having said weight of said examination subject.

7. A method as claimed in claim 1 wherein at least one of said medical examination facilities is an imaging apparatus, and comprising acquiring said third data from meta-information stored together with previously-generated image data from other examination subjects who have been examined using said imaging apparatus.

8. A method as claimed in claim 1 wherein at least some of said medical examination facilities in said plurality of medical examination facilities are magnetic resonance imaging (MRI) facilities, and wherein said third data comprise information selected from the group consisting of information representing availability of different reception coils to receive MRI signals, a table position of an examination bed on which the examination subject lies during an examination with a respective MRI facility, information representing at least one MRI reception coil currently in use at a respective MRI facility to receive MRI signals, information representing a type of MRI measurement currently being implemented by at least one of said MRI facilities, a remaining examination duration of an MRI measurement currently being implemented at one of said MRI facilities, a size of a patient receptacle in a respective MRI facility available to accommodate the examination subject.

9. A method as claimed in claim 8 comprising, in said computer, executing said allocation algorithm to allocate respective examination subjects to respective MRI facilities in order to minimize a number of changes of the MRI reception coils.

10. A method as claimed in claim 8 comprising executing said allocation algorithm to allocate an examination subject exhibiting claustrophobia, or having a weight that is greater than a threshold weight value, to a respective MRI facility that has a larger patient receptacle compared to others of said MRI facilities.

11. A method as claimed in claim 1 comprising, from said computer, accessing a training database that provides said computer with training information representing previous allocations of previous examination subjects to respective medical examination facilities in said plurality of medical examination facilities, said training data informing said computer as to which data, among said first, second and third data, were used in said previous allocations, and wherein said computer executes said allocation algorithm as a learning algorithm using said training data.

12. A method as claimed in claim 1 comprising executing said allocation algorithm in said computer using predetermined rules that designate which medical examination facility, in said plurality of medical examination facilities, should be allocated to a respective examination subject dependent on said first, second and third data.

13. A computer for automatic allocation of examination subjects by a computer of a medical examination site, which has a plurality of medical examination facilities, said method comprising:
a processor;
a communication link with a database via which said processor is configured to access first data from said database, said first data being subject-related data that represent detailed information about an examination subject to be examined using at least one of said plurality of medical examination facilities at said site;
said processor being configured to access said database via said communication link to acquire second data, said second data being operator-related data representing a number of operators working at or available at said plurality of medical examination facilities;
said processor being configured to access said database via said communication link to acquire third data, said third data being medical examination facility-related data representing information about respective medical examination facilities in said plurality of medical examination facilities; and
said processor being configured to execute an allocation algorithm to respectively allocate individual examination subjects to respective medical examination facilities in said plurality of medical examination facilities, dependent on said first data, said second data and said third data, and making a result of execution of said allocation algorithm available from said computer as an output in electronic form.

14. A non-transitory, computer-readable data storage medium for automatic allocation of examination subjects by a computer of a medical examination site, which has a plurality of medical examination facilities, said data storage medium being encoded with programming instructions that, when said storage medium is loaded into a computer, cause said computer to:
access first data from a database, said first data being subject-related data that represent detailed information about an examination subject to be examined using at least one of said plurality of medical examination facilities at said site;
access said database to acquire second data, said second data being operator-related data representing a number of operators working at or available at said plurality of medical examination facilities;
access said database to acquire third data, said third data being medical examination facility-related data representing information about respective medical examination facilities in said plurality of medical examination facilities; and
execute an allocation algorithm to respectively allocate individual examination subjects to respective medical examination facilities in said plurality of medical examination facilities, dependent on said first data, said second data and said third data, and making a result of execution of said allocation algorithm available from said computer as an output in electronic form.
